(19) United States
(12) Patent Application Publication
(10) Pub. No.: US 2021/0012884 A1
(43) Pub. Date: Jan. 14, 2021

(54) AUTOMATED CLASSIFICATION APPARATUS FOR SHOULDER DISEASE VIA THREE DIMENSIONAL DEEP LEARNING METHOD, METHOD OF PROVIDING INFORMATION FOR CLASSIFICATION OF SHOULDER DISEASE AND NON-TRANSITORY COMPUTER READABLE STORAGE MEDIUM OPERATING THE METHOD OF PROVIDING INFORMATION FOR CLASSIFICATION OF SHOULDER DISEASE

(71) Applicant: KOREA INSTITUTE OF SCIENCE AND TECHNOLOGY, Seoul (KR)

(72) Inventors: Youngjun KIM, Seoul (KR); Eungjune SHIM, Seoul (KR); Laehyun KIM, Seoul (KR)

(73) Assignee: KOREA INSTITUTE OF SCIENCE AND TECHNOLOGY, Seoul (KR)

(21) Appl. No.: 16/923,987

(22) Filed: Jul. 8, 2020

(30) Foreign Application Priority Data
Jul. 10, 2019 (KR) 10-2019-0083387

(51) Int. Cl. G16H 30/40; G06K 9/62; G06T 7/00; G06T 3/40

(52) U.S. Cl. CPC G16H 30/40; G06K 9/6268; G06K 2209/05; G06T 7/0012; G06T 3/40; G06K 9/6232

(57) ABSTRACT

An automated classification apparatus includes a 3D (three dimensional) Inception-Resnet block structure, a global average pooling structure and a fully connected layer. The 3D Inception-Resnet block structure includes a 3D Inception-Resnet structure configured to receive 3D medical image of a patient's shoulder and extract features from the 3D medical image and 3D Inception-Downsampling structure configured to downsample information of a feature map including the features. The global average pooling structure is configured to operate an average pooling for an output of the 3D Inception-Resnet block structure. The fully connected layer is disposed after the 3D global average pooling structure. The automated classification apparatus is configured to automatically classify the 3D medical image into a plurality of categories.

### PRIORITY STATEMENT

This application claims priority under 35 U.S.C. § 119 to Korean Patent Application No. 10-2019-0083387, filed on Jul. 10, 2019 in the Korean Intellectual Property Office (KIPO), the contents of which are herein incorporated by reference in their entireties.

### BACKGROUND

### 1. Technical Field

Example embodiments relate to an automated classification apparatus for shoulder disease. More particularly, example embodiments relate to an automated classification apparatus for shoulder disease via a three dimensional (3D) deep learning method.

### 2. Description of the Related Art

Diseases of a shoulder area may be diagnosed by a visual analysis of a three dimensional (3D) medical image such as magnetic resonance imaging or computed tomography imaging by a skilled specialist. It takes a lot of time, effort and experience to effectively analyze the 3D medical image. It is difficult to see a 3D image at a glance in the analysis process, so that the diagnosis may be concluded after repeatedly observing and analyzing multiple 2D images.

In conclusion, in the conventional shoulder disease diagnosis, it may take a lot of time for diagnosis to secure high accuracy, and the result of the diagnosis may depend on a personal skill of the specialist analyzing an image.

### SUMMARY

Example embodiments provide an automated classification apparatus for a shoulder disease capable of automatically classifying a degree of the shoulder disease via a three dimensional deep learning method.

Example embodiments provide a method of providing information of classification of the shoulder disease using the automated classification apparatus for the shoulder disease.

Example embodiments provide a non-transitory computer-readable storage medium having stored thereon program instructions of the method of providing information of classification of the shoulder disease.

In an example automated classification apparatus for a shoulder disease according to the present inventive concept, the automated classification apparatus includes a 3D (three dimensional) Inception-Resnet block structure, a global average pooling structure and a fully connected layer. The 3D Inception-Resnet block structure includes a 3D Inception-Resnet structure configured to receive 3D medical image of a patient's shoulder and extract features from the 3D medical image and 3D Inception-Downsampling structure configured to downsample information of a feature map including the features. The global average pooling structure is configured to operate an average pooling for an output of the 3D Inception-Resnet block structure. The fully connected layer is disposed after the 3D global average pooling structure. The automated classification apparatus is configured to automatically classify the 3D medical image into a plurality of categories.

In an example embodiment, the plurality of the categories may include ‘None’ which means that patient's rotator cuff tear is not present; ‘Partial’, ‘Small’, ‘Medium’ and ‘Large’ according to a size of the patient's rotator cuff tear.

In an example embodiment, the 3D medical image may sequentially pass through a first 3D convolution structure, a first 3D Inception-Resnet block structure, a second 3D Inception-Resnet block structure, a second 3D convolution structure, the global average pooling structure and the fully connected layer.

In an example embodiment, the 3D Inception-Resnet block structure may include three of the 3D Inception-Resnet structures and one of the 3D Inception-Downsampling structure.

In an example embodiment, the 3D Inception-Resnet structure may include a first 3D convolution structure, a second 3D convolution structure and a third 3D convolution structure which are connected in series and forming a first path, a fourth 3D convolution structure and a fifth 3D convolution structure which are connected in series and forming a second path, a first concatenate structure configured to concatenate an output of the third 3D convolution structure and an output of the fifth 3D convolution structure and an add structure configured to operate an element-wise add operation of an output of the first concatenate structure and an input of the 3D Inception-Resnet structure.

In an example embodiment, the 3D Inception-Downsampling structure may include a sixth 3D convolution structure and a maximum pooling structure forming a third path, the maximum pooling structure configured to select a maximum value from the output of the sixth 3D convolution structure, a seventh 3D convolution structure and an average pooling structure forming a fourth path, the average pooling structure configured to select an average value from the output of the seventh 3D convolution structure, a first stride 3D convolution structure including a convolution filter having an increased moving unit and forming a fifth path, a second stride 3D convolution structure different from the first stride 3D convolution structure, including a convolution filter having an increased moving unit and forming a sixth path and a second concatenate structure configured to concatenate an output of the maximum pooling structure, an output of the average pooling structure, an output of the first stride 3D convolution structure and an output of the second stride 3D convolution structure.

In an example embodiment, the automated classification apparatus may further include a region of interest visualization part configured to generate a heat map which visualizes a region of interest identified in the 3D medical image in artificial intelligence generating a diagnostic result of the 3D medical image.

In an example embodiment, the automated classification apparatus may further include a 3D convolution structure disposed between the 3D Inception-Resnet block structure and the global pooling average structure. The region of interest visualization part may be configured to generate the heat map by multiplying first features which are output of the 3D convolution structure and weights learned at the fully connected layer and summing multiplications of the first features and the weights.

In an example embodiment, the heat map may be a 3D class activation map.

In an example method of providing information of classification of shoulder disease according to the present inventive concept, the method includes receiving a 3D (three dimensional) medical image of a patient's shoulder and extracting features from the 3D medical image, using a 3D Inception-Resnet structure, downsampling information of a feature map including the features, using a 3D Inception-Resnet block structure, operating an average pooling for an output of the 3D Inception-Resnet block structure, using a global average pooling structure and automatically classifying the 3D medical image into a plurality of categories.

In an example non-transitory computer-readable storage medium having stored thereon program instructions, the program instructions executable by at least one hardware processor to receive a 3D (three dimensional) medical image of a patient's shoulder and extract features from the 3D medical image, using a 3D Inception-Resnet structure, downsample information of a feature map including the features, using a 3D Inception-Resnet block structure, operate an average pooling for an output of the 3D Inception-Resnet block structure, using a global average pooling structure and automatically classify the 3D medical image into a plurality of categories.

According to the automated classification apparatus for the shoulder disease may receive a 3D medical image and may analyze high dimensional images which a human cannot easily see at a glance using a 3D artificial intelligence algorithm based on a 3D CNN (convolutional neural network). The 3D artificial intelligence algorithm may learn by itself using a large amount of images and big data regarding diagnostic records acquired previously. The 3D artificial intelligence algorithm may represent diagnostic accuracy beyond a skilled orthopedist in a short period.

In addition, the automated classification apparatus for the shoulder disease of the present inventive concept may show a region of interest in medical images as a heat map in addition to accurately diagnosing the shoulder disease. The automated classification apparatus for the shoulder disease of the present inventive concept may generate a 3D class activation map to display regions of interest of the artificial intelligence and provide the 3D class activation map which is rendered in three dimensions as a supplementary information about a diagnosis result.

### BRIEF DESCRIPTION OF THE DRAWINGS

The above and other features and advantages of the present inventive concept will become more apparent by describing in detailed example embodiments thereof with reference to the accompanying drawings, in which:

FIG. 1 is a conceptual diagram illustrating a conventional diagnosis apparatus for a shoulder disease and an automated classification apparatus for the shoulder disease according to an example embodiment of the present inventive concept;

FIG. 2 is a block diagram illustrating a three dimensional (3D) Inception-Downsampling structure according to an example embodiment of the present inventive concept;

FIG. 3 is a block diagram illustrating a 3D Inception-Resnet structure according to an example embodiment of the present inventive concept;

FIG. 4 is a block diagram illustrating a 3D Inception-Resnet block structure according to an example embodiment of the present inventive concept;

FIG. 5 is a block diagram illustrating the automated classification apparatus for the shoulder disease according to an example embodiment of the present inventive concept;

FIG. 6 is a diagram illustrating an operation of a region of interest visualization part of the automated classification apparatus for the shoulder disease according to an example embodiment of the present inventive concept;

FIG. 7 is a screen shot illustrating an operation of the automated classification apparatus for the shoulder disease according to an example embodiment of the present inventive concept;

FIG. 8 is a table illustrating MRI data of rotator cuff used in an example embodiment of the present inventive concept;

FIG. 9 is a table illustrating diagnosis result of the automated classification apparatus for the shoulder disease according to an example embodiment of the present inventive concept, diagnosis result of orthopedists specialized in shoulder and diagnosis result of general orthopedists;

FIGS. 10 and 11 are graphs illustrating diagnosis result of the automated classification apparatus for the shoulder disease according to an example embodiment of the present inventive concept, diagnosis result of orthopedists specialized in shoulder and diagnosis result of general orthopedists;

FIG. 12 is a diagram illustrating how the region of interest changes as artificial intelligence learning progresses in the automated classification apparatus for the shoulder disease according to an example embodiment of the present inventive concept; and

FIG. 13 is a diagram illustrating rotator cuff tear data and region of interest visualization of the automated classification apparatus for the shoulder disease according to an example embodiment of the present inventive concept.

### DETAILED DESCRIPTION OF THE EMBODIMENTS

The present inventive concept now will be described more fully hereinafter with reference to the accompanying drawings, in which exemplary embodiments of the present invention are shown. The present inventive concept may, however, be embodied in many different forms and should not be construed as limited to the exemplary embodiments set fourth herein.

Rather, these exemplary embodiments are provided so that this disclosure will be thorough and complete, and will fully convey the scope of the present invention to those skilled in the art. Like reference numerals refer to like elements throughout.

It will be understood that, although the terms first, second, third, etc. may be used herein to describe various elements, components, regions, layers and/or sections, these elements, components, regions, layers and/or sections should not be limited by these terms. These terms are only used to distinguish one element, component, region, layer or section from another region, layer or section. Thus, a first element, component, region, layer or section discussed below could be termed a second element, component, region, layer or section without departing from the teachings of the present invention.

The terminology used herein is for the purpose of describing particular exemplary embodiments only and is not intended to be limiting of the present invention. As used herein, the singular forms “a,” “an” and “the” are intended to include the plural forms as well, unless the context clearly indicates otherwise. It will be further understood that the terms “comprises” and/or “comprising,” when used in this specification, specify the presence of stated features, integers, steps, operations, elements, and/or components, but do not preclude the presence or addition of one or more other features, integers, steps, operations, elements, components, and/or groups thereof.

Unless otherwise defined, all terms (including technical and scientific terms) used herein have the same meaning as commonly understood by one of ordinary skill in the art to which this invention belongs. It will be further understood that terms, such as those defined in commonly used dictionaries, should be interpreted as having a meaning that is consistent with their meaning in the context of the relevant art and will not be interpreted in an idealized or overly formal sense unless expressly so defined herein.

All methods described herein can be performed in a suitable order unless otherwise indicated herein or otherwise clearly contradicted by context. The use of any and all examples, or exemplary language (e.g., “such as”), is intended merely to better illustrate the invention and does not pose a limitation on the scope of the invention unless otherwise claimed. No language in the specification should be construed as indicating any non-claimed element as essential to the practice of the inventive concept as used herein.

Hereinafter, the present inventive concept will be explained in detail with reference to the accompanying drawings.

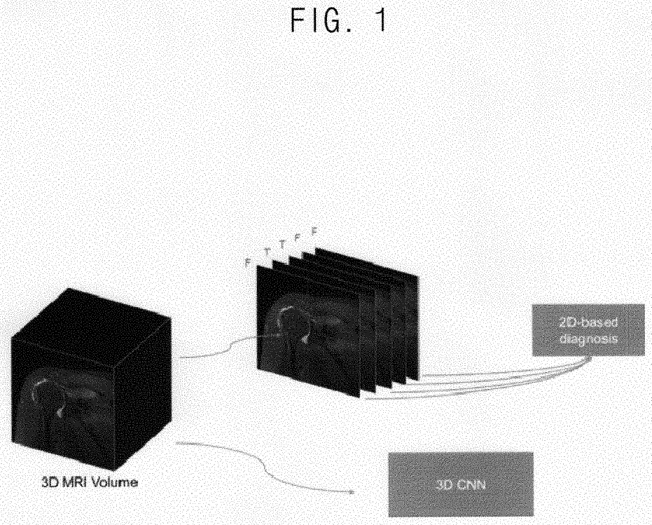

FIG. 1 is a conceptual diagram illustrating a conventional diagnosis apparatus for a shoulder disease and an automated classification apparatus for the shoulder disease according to an example embodiment of the present inventive concept.

Referring to FIG. 1, the conventional diagnosis apparatus for the shoulder disease divides a three dimensional (3D) medical image into a plurality of two dimensional (2D) images for the diagnosis when the 3D medical image is inputted.

The automated classification apparatus for the shoulder disease according to the present example embodiment may receive the 3D medical image, extract features from the 3D medical image, downsample the features and automatically classify the 3D medical image in a plurality of categories as a diagnosis result.

For example, the categories may include “None” which means that a patient's rotator cuff is not ruptured, “Partial”, “Small”, “Medium” and “Large” which mean a degree of the rupture of the patient's rotator cuff.

The automated classification apparatus for the shoulder disease according to the present example embodiment is based on 3D convolutional neural network (CNN). CNN is a deep learning based artificial intelligence algorithm which shows a powerful performance in analyzing images. CNN is a deep learning based algorithm which maximizes the performance of artificial intelligence by deeply connecting an artificial neural network (ANN). CNN includes a lot of learnable convolutional filters for each connection layer so that CNN learns to extract key features of the image from inputted training data. A basic unit of CNN structure is the convolutional filter. By applying a (1*1), (3*3) or (5*5) filter to the 2D image, a meaningful feature may be extracted from the image. In CNN, these filters are filled with initial random values to form a convolutional layer, and as learning progresses, the values of the filters may change to extract the meaningful features. In addition, the convolutional layers are stacked deeply so that the features may be extracted in several stages.

As the convolutional layers are stacked deeply, the donwsampling may be operated by a pooling operation and adjusting a stride value. In the pooling operation, a most significant value is passed to a next layer from a feature map. For example, in a max pooling operation, a maximum value in the feature map may be selected. For example in an average pooling operation, an average value in the feature map may be selected. The stride value may be a parameter of how many pixels the covolutional filter moves when the convolutional filter slides the image.

Through the structure that deeply connects the convolutional layers including these filters, the artificial intelligence may operate a deep learning such that the image is analyzed by utilizing from fine features of a small area of the image to feature of a large area and a desired result is acquired by the analyzing the image. It is the biggest feature and advantage of CNN that CNN analyzes images by viewing such a wide receptive field.

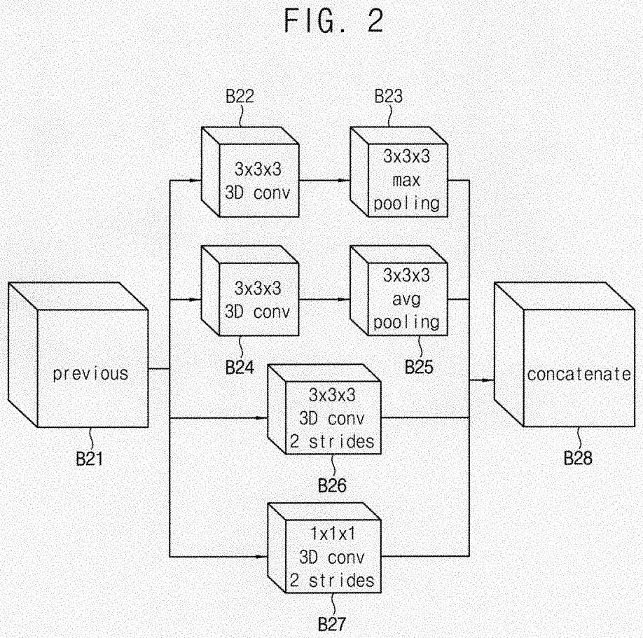

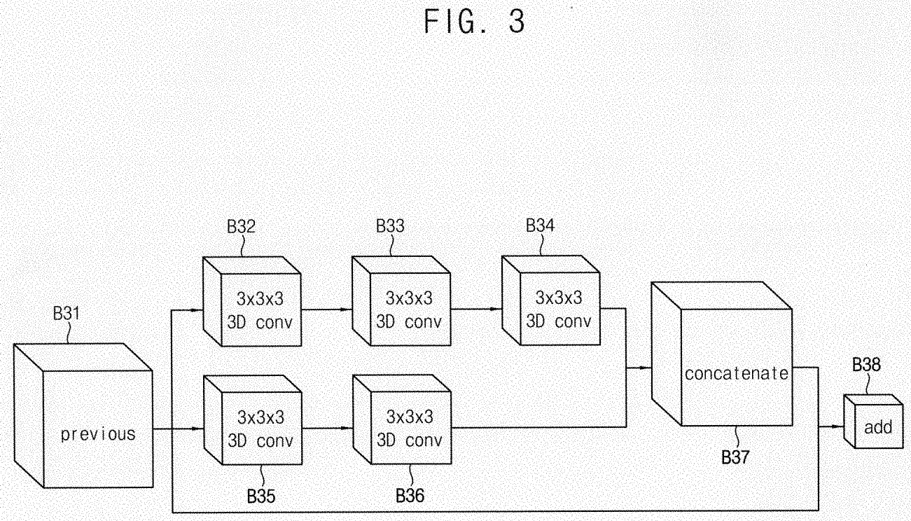

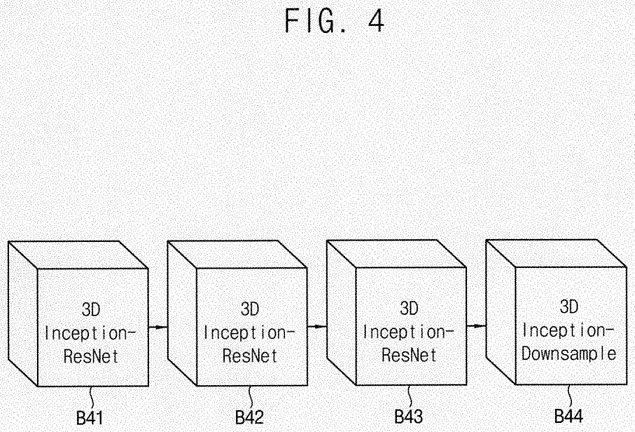

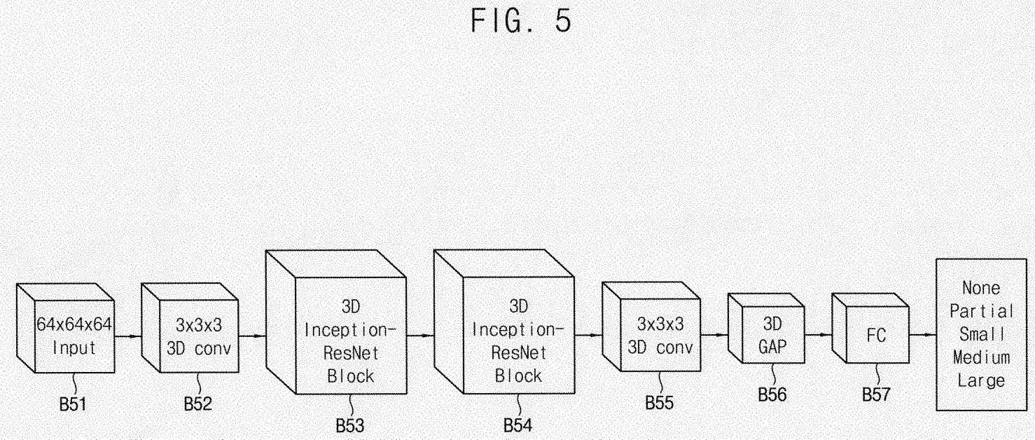

FIG. 2 is a block diagram illustrating a 3D Inception-Downsampling structure according to an example embodiment of the present inventive concept. FIG. 3 is a block diagram illustrating a 3D Inception-Resnet structure according to an example embodiment of the present inventive concept. FIG. 4 is a block diagram illustrating a 3D Inception-Resnet block structure according to an example embodiment of the present inventive concept. FIG. 5 is a block diagram illustrating the automated classification apparatus for the shoulder disease according to an example embodiment of the present inventive concept.

Referring to FIGS. 2 to 5, the automated classification apparatus for the shoulder disease includes the 3D Inception-Resnet block structures B53 and B54, a 3D global average pooling structure 3D GAP and B56 and a fully connected layer FC and B57 disposed after the 3D global average pooling structure B56.

For example, in the automated classification apparatus for the shoulder disease, the 3D medical image B51 may sequentially pass through a first 3D convolution structure B52, a first 3D convolution structure B52, a first 3D Inception-Resnet block structure B53, a second 3D Inception-Resnet block structure B54, a second 3D convolution structure B55, the 3D global average pooling structure B56 and the fully connected layer B57. In the present example embodiment, the 3D medical image B51 may be a 64*64*64 input image.

The 3D Inception-Resnet block structure B53 and B54 may include three of the 3D Inception-Resnet structures B41, B42 and B43 and one of the 3D Inception-Downsampling structure B44 which are connected in series. The three of the 3D Inception-Resnet structures B41, B42 and B43 may have the same structure. Alternatively, the three of the 3D Inception-Resnet structures B41, B42 and B43 may have different structures from one another.

The 3D Inception-Resnet structure (at least one of B41, B42 and B43) may include a first 3D convolution structure B32, a second 3D convolution structure B33 and a third 3D convolution structure B34 connected in series and forming a first path, a fourth 3D convolution structure B34 and a fifth 3D convolution structure B36 connected in series and forming a second path, a concatenate structure B37 concatenating an output of the third 3D convolution structure B34 and an output of the fifth 3D convolution structure B36 and an add structure B38 operating an element-wise add operation of the input of the 3D

Inception-Resnet structure and an output of the concatenate structure B37.

The first 3D convolution structure B32 and the fourth 3D convolution structure B34 are connected to a previous block B31 and receive the input of the 3D Inception-Resnet structure B41, B42 and B43.

The 3D Inception-Downsampling structure B44 may include a first 3D convolution structure B22 and a maximum pooling structure B23 forming a first path. The maximum pooling structure B23 may select a maximum value in the output of the first 3D convolution structure B22. The 3D Inception-Downsampling structure B44 may further include a second 3D convolution structure B24 and an average pooling structure B25 forming a second path. The average pooling structure B25 may select an average value in the output of the second 3D convolution structure B24. The 3D Inception-Downsampling structure B44 may further include a first stride 3D convolution structure B26 including a convolution filter having an increased moving unit and forming a third path. The 3D Inception-Downsampling structure B44 may further include a second stride 3D convolution structure B27 including a convolution filter having an increased moving unit, different from the first stride 3D convolution structure B26 and forming a fourth path. The 3D Inception-Downsampling structure B44 may further include a concatenate B28 concatenating an output of the maximum pooling structure B23, an output of the average pooling structure B25, an output of the first stride 3D convolution structure B26 and an output of the second stride 3D convolution structure B27.

The first stride 3D convolution structure B26 may be a 3*3*3 3D convolution structure. The stride of the first stride 3D convolution structure B26 which means the moving unit of the convolution filter may be two. The second stride 3D convolution structure B27 may be a 1*1*1 3D convolution structure. The stride of the second stride 3D convolution structure B27 which means the moving unit of the convolution filter may be two.

The first 3D convolution structure B22, the second 3D convolution structure B24, the first stride 3D convolution structure B26 and the second stride 3D convolution structure B27 are connected to a previous block B21 and receive the input of the 3D Inception-Downsampling structure B44.

Referring again to FIG. 2, the 3D Inception-Downsampling structure B44 extracts the feature of the previous volume B21 and generates the downsampled output. In the 3D Inception-Downsampling structure B44, the downsampling is operated using the pooling and the stride. By pooling, the significant one is selected among the features which are the result of the convolution. The moving unit of the convolution filter is increased by the stride, the size of the output may be reduced than the original image.

The results of the downsampled by each method may be all set to have the same size, so the results are concatenated (B37) like stacking the papers and the concatenated result are transmitted to a next layer. The 3D Inception-Downsampling structure B44 generates a lot of output features having the reduced size than the previous block B21 so that the result of the 3D Inception-Downsampling structure B44 may be the contracted information for a larger range.

Referring again to FIG. 3, the 3D Inception-Resnet structure (at least one of B41, B42 and B43) is implemented using a 3D convolution filter. The 3D Inception-Resnet structure includes a various types of the convolution filters B32, B33, B34, B35 and B36 extracting meaningful information from the image received from the previous block B31 or the feature map. In the 3D Inception-Resnet structure, the size of the output passing through each of the (3*3*3) filters may be same as the size of the input. In the concatenate structure B37, two different structures are concatenated so that the features having more various forms may be extracted.

Referring again to FIG. 4, the 3D Inception-Resnet block structure B53 and B54 includes the three 3D Inception-Resnet structures B41, B42 and B43 at a front and the single 3D Inception-Downsampling structure B44 at the last. Via the structures of FIG. 3 and the structure of FIG. 2, the output may be generated by contracting (downsampling) the input image or the input feature map.

Referring again to FIG. 5, an entire network structure of the automated classification apparatus for the rotator cuff tear may include two 3D Inception-Resnet block structures B53 and B54. Each of the 3D Inception-Resnet block structures B53 and B54 may have the structure of FIG. 4. The 3D Inception-Resnet block structures B53 and B54 learn filters which may extract a lot of 3D features which are analyzed in 3D. When the input image B51 passes through the (3*3*3) convolution layers B52 and B55 or the 3D Inception-Resnet block structures B53 and B54, the information of the input image B51 may be contracted and the meaningful features may be extracted so that the final decision may be determined. The single convolution filters B52 and B55 and the 3D Inception-Resnet block structures B53 and B54 basically have a common characteristic that extracts features from the image, but the 3D Inception-Resnet block structures B53 and B54 may obtain more information than the single convolution filters B52 and B55.

Most CNN applied studies are based on 2D images, and practically, a lot of input data are 2D images. However, the medical image such as CT or MRI is a 3D volume image that has image information inside the patient's body. A lot of medical image analysis studies using CNN-based algorithm are also actively performed, but it is not possible to fully use the rich information of the 3D image because of using the method of analyzing multiple 2D images.

In the present example embodiment, the reading of MRI images is trained using the 3D Inception-Resnet structure capable of extracting the 3D features from the image by extending the above-mentioned convolution filter of CNN in a three dimension.

The conventional CNN method may have a structure of simply layering the convolution layers. In contrast, the present Inception-Resnet structure may combine the structure of Inception structure and Resnet structure to the convolution layers. In the Inception structure, outputs of the different convolution filters disposed in parallel are concatenated. The Inception structure may represent better results in terms of both a calculation quantity and a performance compared to stacking the same number of filters in the conventional method. In the Resnet structure, the output of passing through the several convolution filters and the image of the previous stage are element-wise added by a residual block so that the performance of the CNN may be enhanced by keeping the information close to the original image in the previous stage.

In the proposed 3D Inception-Resnet structure, the convolution filter, which is the basic unit, is extended to 3D to extract features from the 3D volume. The proposed 3D Inception-Resnet structure includes the (1*1*1) filter and the (3*3*3) filter and downsamples the feature map by pooling and stride adjustment. The proposed 3D Inception-Resnet structure may include the 3D Inception-Resnet structure B41, B42 and B43 and the 3D Inception-Downsample structure B44. The 3D Inception-Resnet block structure B53 and B54 may be generated by combining the 3D Inception-Resnet structure B41, B42 and B43 and the 3D Inception-Downsample structure B44. The entire network structure of the automated classification apparatus for the rotator cuff tear may be generated using two 3D Inception-Resnet structure B41, B42 and B43 and the 3D Inception-Downsample structure B44.

In order to calculate the 3D Class Activation Map (CAM), which will be described later, the global average pooling (GAP) layer B56 and a single fully-connected (FC) layer B57 may be disposed at the last stage. The GAP layer B56 calculates an average of each of the feature maps of the output of the last convolution layer. By the GAP layer B56, a weight in each position may be estimated. The FC layer B57 learns parameters for a final classification using the output of the GAP layer B56. Although the performance may be enhanced when the plural FC layers B57 are used, a location information may be lost while passing the plural FC layers B57. Thus, the single FC layer B57 is used in the present example embodiment for the CAM calculation. When the number of the FC layer B57 is little, the amount of computation may be reduced so that it may be efficient in the amount of computation.

According to the present example embodiment, by applying the above explained methods, the performance of CNN shown in the 2D image may be extended to the 3D image. Since the present example embodiment may efficiently analyze the 3D image of the patient having the large receptive field, the efficiency of time and cost may be enhanced rather than actually making a diagnosis in the medical field as well as rather than the conventional methods.

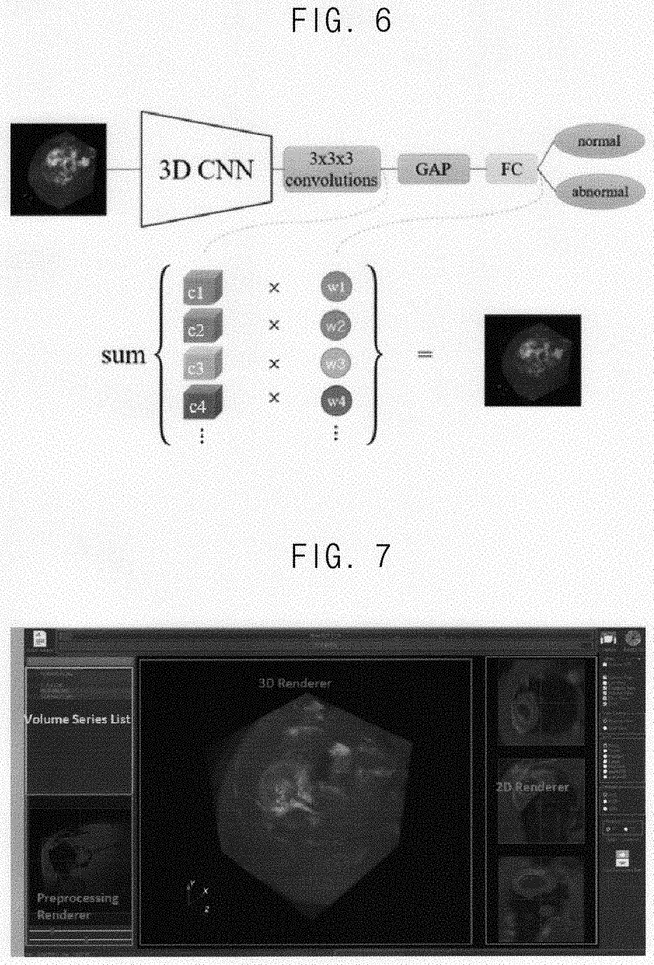

FIG. 6 is a diagram illustrating an operation of a region of interest visualization part of the automated classification apparatus for the shoulder disease according to an example embodiment of the present inventive concept.

Referring to FIGS. 1 to 6, the automated classification apparatus for the shoulder disease may further include the region of interest visualization part generating a heat map visualizing the region of interest identified in the 3D medical image in artificial intelligence generating diagnostic results of the 3D medical image.

The region of interest visualization part may generate the heat map by multiplying the features c1, c2, c3, c4, . . . which are the output of the second 3D convolution structure B55 and the weights w1, w2, w3, w4, . . . learned at the fully connected layer B57 and summing the multiplication of the features c1, c2, c3, c4, . . . and the weights w1, w2, w3, w4, . . . . For example, the heat map may be a 3D class activation map.

FIG. 6 illustrates extraction of the region of interest from an original image by the class activation map calculation. The 3D heat map are generated by summing the multiplication of the feature maps c1, c2, c3, c4, . . . output from the last convolution and the weights w1, w2, w3, w4, . . . learned at the FC layer.

After the CNN is learned, the feature areas, which the artificial intelligence has seen as significant when making decisions, may be visualized using the class activation map method.

Since CNN learned to extract many features internally, the visualization may be possible using the image for making decisions and the learned filter in late layers of the CNN structure. In the case of medical imaging diagnosis, the visualization of the region of interest is important because it is clinically important to explain detailed diagnosis results beyond simple diagnosis prediction. In the present example embodiment, the 3D CNN is used so that the class activation map may be calculated in 3D and the 3D visualization may be possible. By visualizing the region of interest with MRI data, it is possible to see which region is important for predictions made by artificial intelligence.

This visualization not only improves the reliability of the learning and prediction results, but also predicts where the problem occurred clinically.

FIG. 7 is a screen shot illustrating an operation of the automated classification apparatus for the shoulder disease according to an example embodiment of the present inventive concept.

Referring to FIGS. 1 to 7, when the integrated software which is effective for the learned 3D CNN actual medical field is used, it is possible to use AI-based diagnostics with proven reliability and stability regardless of time and place.

The software has functions for importing medical data, performing 2D and 3D visualization, performing AI-based diagnostics, and visualizing the region of interest. The importing function reads a Dicom file (having an extension of *.dcm), an image format commonly used in medical images, to reconstruct image and 3D visualization information. When a user only has a MRI data on the shoulder, the user may check the patient's presence of rotator cuff tears in real time and may receive the 3D visualized information by simply selecting the largest bone in the shoulder, Humerus, with the mouse, without prior medical knowledge.

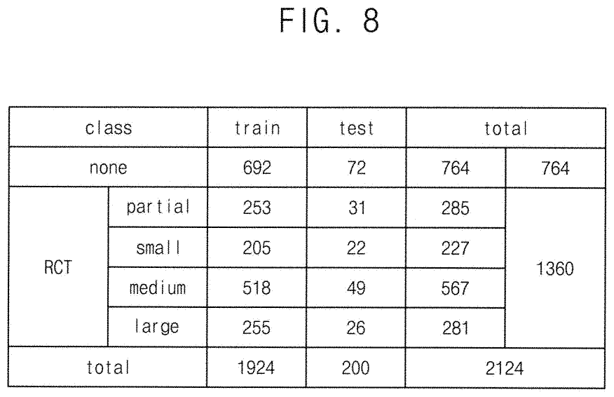

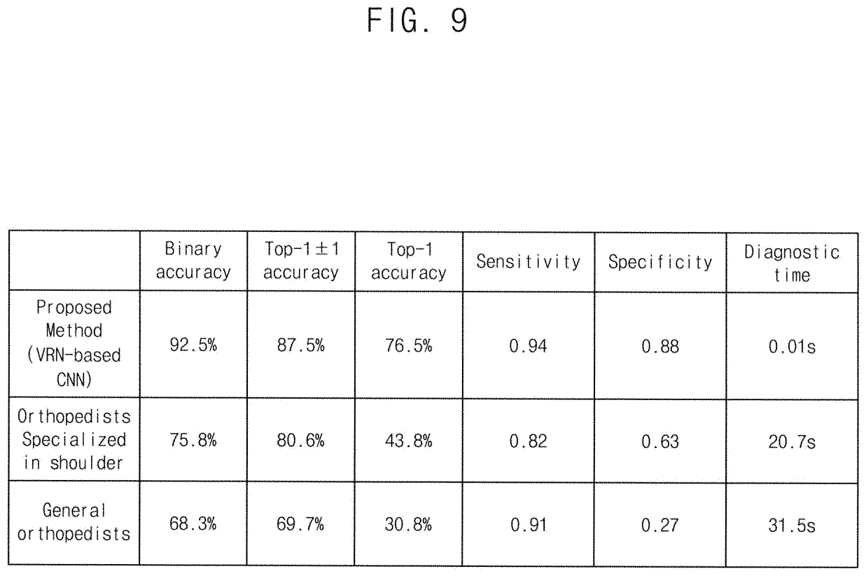

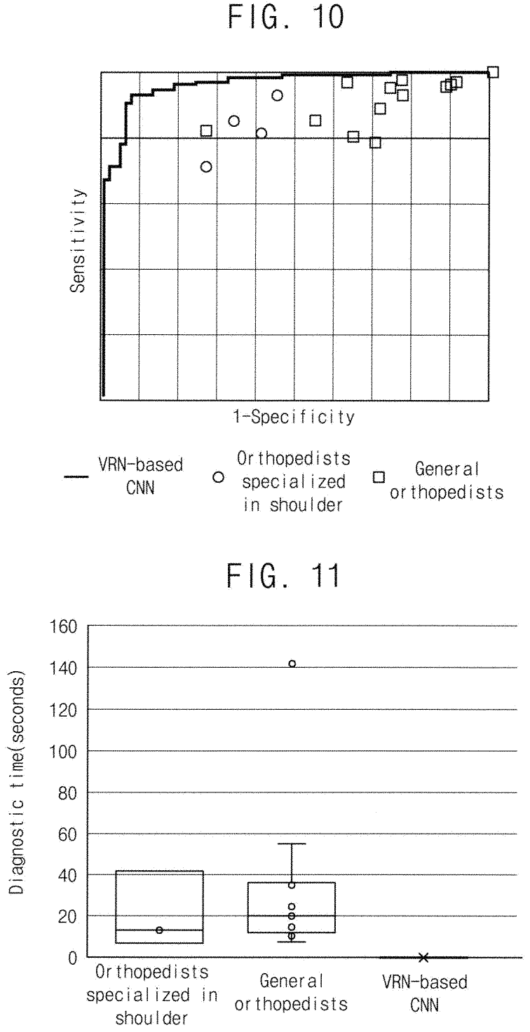

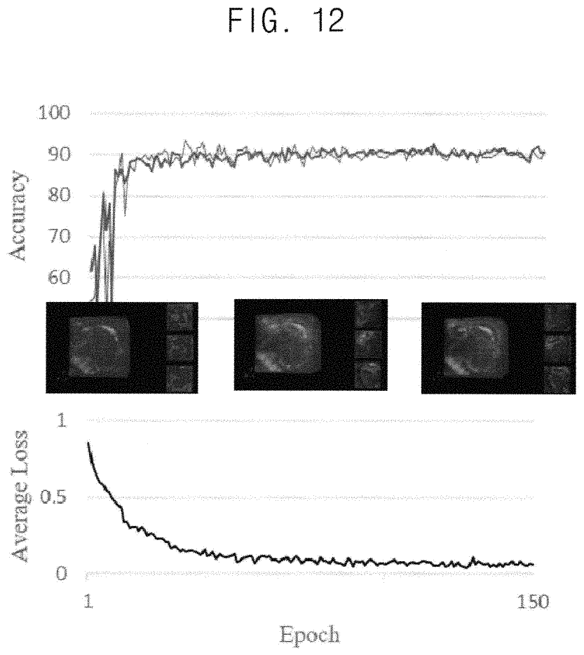

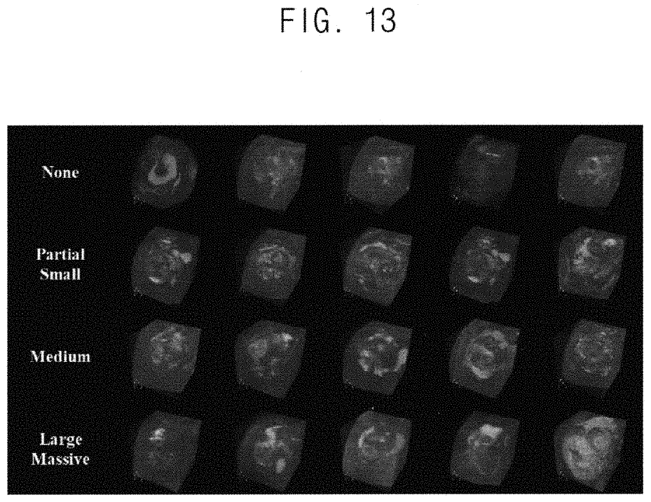

FIG. 8 is a table illustrating MRI data of rotator cuff used in an example embodiment of the present inventive concept. FIG. 9 is a table illustrating diagnosis result of the automated classification apparatus for the shoulder disease according to an example embodiment of the present inventive concept, diagnosis result of orthopedists specialized in shoulder and diagnosis result of general orthopedists. FIGS. 10 and 11 are graphs illustrating diagnosis result of the automated classification apparatus for the shoulder disease according to an example embodiment of the present inventive concept, diagnosis result of orthopedists specialized in shoulder and diagnosis result of general orthopedists. FIG. 12 is a diagram illustrating how the region of interest changes as artificial intelligence learning progresses in the automated classification apparatus for the shoulder disease according to an example embodiment of the present inventive concept. FIG. 13 is a diagram illustrating rotator cuff tear data and region of interest visualization of the automated classification apparatus for the shoulder disease according to an example embodiment of the present inventive concept.

Referring to FIGS. 1 to 13, for rotator cuff tear MRI data, a performance test of the automated classification apparatus for the shoulder disease according to the present example embodiment was performed. A total of 2124 MRI images representing presences of tear and sizes of tears are used. Of the 2124 MRI images, 200 randomly sampled data were designated as test data, and the remaining 1,924 patient data were used for learning. For the 200 test data, the actual diagnosis was performed by 4 orthopedists specialized in shoulder and 13 general orthopedists to test the accuracy between the automated classification apparatus for the shoulder disease according to the present example embodiment and the orthopedists specialized in shoulder and the general orthopedists. The artificial intelligence learning classified the images into five categories (None, Partial, Small, Medium, Large) based on the presence of the rotator cuff tear and the sizes of the rotator cuff tears.

As a result of the experiment, the automated classification apparatus for the shoulder disease according to the present example embodiment represents an accuracy of 76.5% in a case of accurately predicting the size of the rotator cuff tear (Top-1 accuracy). The orthopedists specialized in shoulder represents an accuracy of 43.8% and the general orthopedists represents an accuracy of 30.8% in the Top-1 accuracy so that the Top-1 accuracy of the automated classification apparatus for the shoulder disease according to the present example embodiment was higher than the Top-1 accuracy of the orthopedists specialized in shoulder by 32.7% and than the Top-1 accuracy of the general orthopedists by 45.7%

The automated classification apparatus for the shoulder disease according to the present example embodiment represents an accuracy of 92.5% in a case of predicting only the presence of the rotator cuff tear (Binary accuracy). The orthopedists specialized in shoulder represents an accuracy of 75.8% and the general orthopedists represents an accuracy of 68.3% in the Binary accuracy so that the Binary accuracy of the automated classification apparatus for the shoulder disease according to the present example embodiment was higher than the Binary accuracy of the orthopedists specialized in shoulder by 16.7% and than the Binary accuracy of the general orthopedists by 24.2%

In an aspect of diagnosis time, the automated classification apparatus for the shoulder disease according to the present example embodiment represents high efficiency. It shows that the time required to diagnose all 200 patient data can be accurately diagnosed in real time with 0.01 seconds per person by the automated classification apparatus for the shoulder disease according to the present example embodiment. An average of 20.7 seconds were required to read one person's data for the orthopedists specialized in shoulder. An average of 31.5 seconds were required to read one person's data for the general orthopedists.

As shown in FIG. 12, it is possible to check how the region of interest changes with 3D visualization data using the 3D CAM method as artificial intelligence learning progresses (as epoch increases).

FIG. 13 represents the data (None, Partial, Small, Medium, Large) including the presence of the rotator cuff tear and the size of the rotator cuff tear with the region of interest visualization. By the region of interest visualization data, the reliability of learning and prediction results may be improved and it is possible to predict where clinically the problem occurred.

The present inventive concept is related to the automated classification apparatus for the shoulder disease and the visualization apparatus using 3D deep learning, the diagnosis accuracy may be enhanced and the diagnosis time and the diagnosis cost may be reduced.

The foregoing is illustrative of the present inventive concept and is not to be construed as limiting thereof. Although a few example embodiments of the present inventive concept have been described, those skilled in the art will readily appreciate that many modifications are possible in the example embodiments without materially departing from the novel teachings and advantages of the present inventive concept. Accordingly, all such modifications are intended to be included within the scope of the present inventive concept as defined in the claims. In the claims, means-plus-function clauses are intended to cover the structures described herein as performing the recited function and not only structural equivalents but also equivalent structures. Therefore, it is to be understood that the foregoing is illustrative of the present inventive concept and is not to be construed as limited to the specific example embodiments disclosed, and that modifications to the disclosed example embodiments, as well as other example embodiments, are intended to be included within the scope of the appended claims. The present inventive concept is defined by the following claims, with equivalents of the claims to be included therein.

What is claimed is:

1. An automated classification apparatus for a shoulder disease comprising:
a 3D (three dimensional) Inception-Resnet block structure comprising a 3D Inception-Resnet structure configured to receive 3D medical image of a patient's shoulder and extract features from the 3D medical image and 3D Inception-Downsampling structure configured to downsample information of a feature map including the features; and
a global average pooling structure configured to operate an average pooling for an output of the 3D Inception-Resnet block structure; and
a fully connected layer disposed after the 3D global average pooling structure,
wherein the automated classification apparatus is configured to automatically classify the 3D medical image into a plurality of categories.

2. The automated classification apparatus of claim 1, wherein the plurality of the categories includes ‘None’ which means that patient's rotator cuff tear is not present; ‘Partial’, ‘Small’, ‘Medium’ and ‘Large’ according to a size of the patient's rotator cuff tear.

3. The automated classification apparatus of claim 1, wherein the 3D medical image sequentially passes through a first 3D convolution structure, a first 3D Inception-Resnet block structure, a second 3D Inception-Resnet block structure, a second 3D convolution structure, the global average pooling structure and the fully connected layer.

4. The automated classification apparatus of claim 1, wherein the 3D Inception-Resnet block structure comprises three of the 3D Inception-Resnet structures and one of the 3D Inception-Downsampling structure.

5. The automated classification apparatus of claim 1, wherein the 3D Inception-Resnet structure comprises:
a first 3D convolution structure, a second 3D convolution structure and a third 3D convolution structure which are connected in series and forming a first path;
a fourth 3D convolution structure and a fifth 3D convolution structure which are connected in series and forming a second path;
a first concatenate structure configured to concatenate an output of the third 3D convolution structure and an output of the fifth 3D convolution structure; and
an add structure configured to operate an element-wise add operation of an output of the first concatenate structure and an input of the 3D Inception-Resnet structure.

6. The automated classification apparatus of claim 5, wherein the 3D Inception-Downsampling structure comprises:
a sixth 3D convolution structure and a maximum pooling structure forming a third path, the maximum pooling structure configured to select a maximum value from the output of the sixth 3D convolution structure;
a seventh 3D convolution structure and an average pooling structure forming a fourth path, the average pooling structure configured to select an average value from the output of the seventh 3D convolution structure;
a first stride 3D convolution structure including a convolution filter having an increased moving unit and forming a fifth path;
a second stride 3D convolution structure different from the first stride 3D convolution structure, including a convolution filter having an increased moving unit and forming a sixth path; and
a second concatenate structure configured to concatenate an output of the maximum pooling structure, an output of the average pooling structure, an output of the first stride 3D convolution structure and an output of the second stride 3D convolution structure.

7. The automated classification apparatus of claim 1, further comprising a region of interest visualization part configured to generate a heat map which visualizes a region of interest identified in the 3D medical image in artificial intelligence generating a diagnostic result of the 3D medical image.

8. The automated classification apparatus of claim 7, further comprising a 3D convolution structure disposed between the 3D Inception-Resnet block structure and the global pooling average structure,
wherein the region of interest visualization part is configured to generate the heat map by multiplying first features which are output of the 3D convolution structure and weights learned at the fully connected layer and summing multiplications of the first features and the weights.

9. The automated classification apparatus of claim 8, wherein the heat map is a 3D class activation map.

10. A method of providing information for classification of shoulder disease, the method comprising:
receiving a 3D (three dimensional) medical image of a patient's shoulder and extracting features from the 3D medical image, using a 3D Inception-Resnet structure;
downsampling information of a feature map including the features, using a 3D Inception-Resnet block structure;
operating an average pooling for an output of the 3D Inception-Resnet block structure, using a global average pooling structure; and
automatically classifying the 3D medical image into a plurality of categories.

11. A non-transitory computer-readable storage medium having stored thereon program instructions, the program instructions executable by at least one hardware processor to:
receive a 3D (three dimensional) medical image of a patient's shoulder and extract features from the 3D medical image, using a 3D Inception-Resnet structure;
downsample information of a feature map including the features, using a 3D Inception-Resnet block structure;
operate an average pooling for an output of the 3D Inception-Resnet block structure, using a global average pooling structure; and
automatically classify the 3D medical image into a plurality of categories.
